(19) United States
(12) Patent Application Publication
(10) Pub. No.: US 2023/0302258 A1
(43) Pub. Date: Sep. 28, 2023

(54) CATHETER

(71) Applicant: KANEKA CORPORATION, Osaka-shi, Osaka (JP)

(72) Inventor: Toshiya KISA, Okaya-shi (JP)

(73) Assignee: KANEKA CORPORATION, Osaka-shi, Osaka (JP)

(21) Appl. No.: 17/928,720

(22) PCT Filed: May 27, 2021
(86) PCT No.: PCT/JP2021/020269

§ 371 Date: Jun. 7, 2023

(30) Foreign Application Priority Data
Jun. 4, 2020 (JP) 2020-097338

(51) Int. Cl. A61M 25/01

(52) U.S. Cl. CPC A61M 25/0147; A61M 25/0136; A61M 2205/0216

(57) ABSTRACT

The objective is to provide an asymmetrically bendable catheter configured such that a bent shape is different depending on whether the catheter distal part bends to one side or the other side. Disclosed is a catheter having a shaft, a first wire, a second wire, a leaf spring disposed in the lumen of the shaft so as to separate the lumen of the shaft into a first part where the first wire is disposed and a second part where the second wire is disposed, a supporting member, and a first coil having a lumen in which the first wire is disposed; the first coil fixed to a proximal end side of the leaf spring at two or more locations, and the first coil having a total length L₁ in its natural state and a total length L_{C1} at maximum compression, and a ratio L_{C1}/L₁ is 0.9 or more.

### TECHNICAL FIELD

The present invention relates to a catheter with a bendable distal part.

### BACKGROUND ART

Electrode catheter with multiple electrodes on the distal part are used to measure or pace potentials in the heart. Some such catheters with electrodes have a distal part that is bendable by operating a handle so that the distal part of the catheter can be easily placed at a desired site in the heart. Such catheters generally allow the distal part of the catheter to be curved by pulling a pull wire fixed to the inside of the distal end part of the catheter. In addition, catheters with two pull wires have been proposed to bend the catheter on both one side and the other side with respect to the longitudinal direction of the catheter.

For example, Patent document 1 discloses a tip deflection operable catheter with a plurality of tubes for manipulating in which manipulation wires are arranged in the lumen of the catheter tube, and the tubes for manipulating are divided into a plurality of partial tubes so that the tip part of the catheter can be deformed into a smooth curved shape without bending in the middle. Furthermore, tip deflection operable catheters that can prevent the tip electrodes, the leaf spring, or pull wires placed inside the distal end part of the catheter from falling out even when the tip of the catheter is curved (Patent documents 2, 3), and catheters with different shapes of curvature on one side and the other side have also been proposed (Patent documents 4, 5).

### RELATED ART DOCUMENT

### Patent Document

Patent document 1: JP 2012-200445 A
Patent document 2: JP 2010-75530 A
Patent document 3: JP 2015-100515 A
Patent document 4: JP 2012-147971 A
Patent document 5: JP 2017-518122 T

### SUMMARY OF THE INVENTION

### Problems to be Solved by the Invention

In order to deliver the distal part of the catheter to the optimal site according to the size of the heart and purposes, it is required for the distal part of the catheter to not only be able to be curved to both one side and the other side, but also be an asymmetrically curved type catheter with different diameters of curvature for each side. In addition, by increasing the degree of asymmetry of the curvature, the distal part of the catheter can be easily delivered to the desired site. However, it was difficult to make a catheter with a higher degree of asymmetry of the curvature diameter in the configuration as disclosed in Patent documents 4 and 5.

The present invention has been made in view of the above circumstances, and its objective is to provide an asymmetrically curved type catheter in which the distal part of the catheter can be curved to both one side and the other side, and the respective curved shapes are different.

### Means for Solving the Problems

A catheter that can solve the above problems has a shaft having a distal end, a proximal end, and a lumen extending in a longitudinal direction; a first wire having a distal end and a proximal end and extending in the lumen of the shaft, the distal end being fixed to a distal end part of the shaft, and the proximal end being disposed at a proximal end part of the shaft; a second wire having a distal end and a proximal end and extending in the lumen of the shaft, the distal end being fixed to a distal end part of the shaft, and the proximal end being disposed at a proximal end part of the shaft; a leaf spring disposed in the lumen of the shaft so as to separate the lumen of the shaft into a first part where the first wire is disposed and a second part where the second wire is disposed in the longitudinal direction; a supporting member, to which the proximal end of the leaf spring is fixed, extending in the longitudinal direction, having a lumen in which the first wire and the second wire are disposed, and being disposed proximal to the leaf spring; and a first coil having a lumen in which the first wire is disposed and being disposed in the first part and distal to a distal end of the supporting member, wherein the first coil is fixed to a proximal end side of the leaf spring at two or more locations, and has a first fixed portion where the first coil and the leaf spring are fixed, a second fixed portion where the first coil and the leaf spring are fixed, and an intermediate unfixed portion where the first coil and the leaf spring are not fixed; the second fixed portion is located proximal to the first fixed portion; the intermediate unfixed portion is located between the first fixed portion and the second fixed portion; and the first coil has a total length L₁ in its natural state and a total length L_{C1} at maximum compression, and a ratio L_{C1}/L₁ is 0.9 or more.

Preferably, the supporting member is a proximal tube.

Preferably, the first coil is uncompressed.

Preferably, the portion where the first coil and the leaf spring are fixed is located on a side of the first coil facing a first side of the leaf spring.

Preferably, an unfixed portion having a longest length in the longitudinal direction among unfixed portions has a length in its natural state in the longitudinal direction of 50% or longer of the total length L₁ of the first coil in its natural state.

Preferably, the first coil further has a distal unfixed portion that does not include fixed portion where the first coil and the leaf spring is fixed between a distal end of the first coil and the first fixed portion.

Preferably, the catheter further has a second coil having a lumen in which the first wire is disposed and being disposed in the first part and distal to the first coil.

In this case, the second coil preferably has a total length L₂ in its natural state and a total length L_{C2} at maximum compression, and the ratio L_{C2}/L₂ is less than 0.9.

Preferably, a bending stiffness of the first coil is greater than a bending stiffness of the second coil, and the difference between the bending stiffness of the first coil and the bending stiffness of the second coil is 50% or less.

Preferably, the first coil includes a helically wound first coil wire, the second coil includes a helically wound second coil wire, and a pitch interval of the first coil is shorter than a pitch interval of the second coil.

Preferably, a coil wire diameter of the first coil is the same as a coil wire diameter of the second coil, and a coil diameter of the first coil is the same as a coil diameter of the second coil.

Preferably, the catheter further has a third coil having a lumen in which the second wire is disposed and being disposed in the second part.

Preferably, the third coil has a total length L₃ in its natural state and a total length L_{C3} at maximum compression, and the ratio L_{C3}/L₃ is less than 0.9.

Preferably, the catheter has a second coil having a lumen in which the first wire is disposed and being disposed in the first part and distal to the first coil, a third coil having a lumen in which the second wire is disposed and being disposed in the second part, and a protection tube having a lumen and being disposed in the lumen of the shaft, wherein the leaf spring, the first coil, the second coil, and the third coil are placed in the lumen of the protection tube.

Preferably, the first coil is fixed by welding, soldering, bonding, or crimping at the first fixed part and the second fixed part.

### Effects of the Invention

The present invention can provide an asymmetrically curved type catheter in which the distal part of the catheter can be curved on both one side and the other side, each with a different curved shape, thus allowing the distal part of the catheter to be easily delivered to the desired location.

### BRIEF DESCRIPTION OF THE DRAWINGS

FIG. 1 is a plan view of a catheter according to an embodiment of the present invention.

FIG. 2 is a plan view of a catheter according to an embodiment of the present invention.

FIG. 3 is a plan view of a catheter according to an embodiment of the present invention.

FIG. 4 is a cross-sectional view of the distal part of the catheter shown in FIG. 2 in the longitudinal direction.

FIG. 5 is a V-V cross-sectional view of the distal part of the catheter shown in FIG. 4.

FIG. 6 is a VI-VI cross-sectional view of the distal part of the catheter shown in FIG. 4.

FIG. 7 is a VII-VII cross-sectional view of the distal part of the catheter shown in FIG. 4.

FIG. 8 is a side view of the shaft interior of part A of the distal part of the catheter shown in FIG. 4.

FIG. 9 is a side view of a first coil according to an embodiment of the present invention.

FIG. 10 is a side view of the first coil shown in FIG. 9 at maximum compression.

FIG. 11 is a side view of a first coil according to another embodiment of the present invention.

FIG. 12 is a cross-sectional view of a distal part of a catheter according to another embodiment of the present invention in the longitudinal direction.

FIG. 13 is a cross-sectional view of a distal part of a catheter according to still another embodiment of the present invention in the longitudinal direction.

FIG. 14 shows a stiffness measurement method.

FIG. 15 shows a different example of the V-V cross-sectional view of the distal part of the catheter shown in FIG. 4.

### MODE FOR CARRYING OUT THE INVENTION

Hereinafter, the present invention will be described based on the following embodiments, however, the present invention is not limited by the following embodiments and can be altered in design within a scope in compliance with the intent described above and below, and all the changes are to be encompassed within a technical scope of the present invention. Note that, in each drawing, hatching, reference signs for components, and the like may be omitted for convenience of description, and in such a case, the specification and other drawings are to be referred to. Furthermore, since the dimensions of the various components in the drawings are provided for the purpose of facilitating the understanding of the feature of the present invention, the dimensions may differ from the actual dimensions in some cases.

A catheter of the present invention has a shaft having a distal end, a proximal end, and a lumen extending in a longitudinal direction; a first wire having a distal end and a proximal end and extending in the lumen of the shaft, the distal end being fixed to a distal end part of the shaft, and the proximal end being disposed at a proximal end part of the shaft; a second wire having a distal end and a proximal end and extending in the lumen of the shaft, the distal end being fixed to a distal end part of the shaft, and the proximal end being disposed at a proximal end part of the shaft; a leaf spring disposed in the lumen of the shaft so as to separate the lumen of the shaft into a first part where the first wire is disposed and a second part where the second wire is disposed in the longitudinal direction; a supporting member, to which the proximal end of the leaf spring is fixed, extending in the longitudinal direction, having a lumen in which the first wire and the second wire are disposed, and being disposed proximal to the leaf spring; and a first coil having a lumen in which the first wire is disposed and being disposed in the first part and distal to a distal end of the supporting member, wherein the first coil is fixed to a proximal end side of the leaf spring at two or more locations, and has a first fixed portion where the first coil and the leaf spring are fixed, a second fixed portion where the first coil and the leaf spring are fixed, and an intermediate unfixed portion where the first coil and the leaf spring are not fixed; the second fixed portion is located proximal to the first fixed portion; the intermediate unfixed portion is located between the first fixed portion and the second fixed portion; and the first coil has a total length L₁ in its natural state and a total length L_{C1} at maximum compression, and a ratio L_{C1}/L₁ is 0.9 or more.

With the above configuration, the catheter of the present invention can be an asymmetrically curved type catheter where the distal part of the catheter can be curved to both one side and the other side of the leaf spring in the radial direction of the shaft, and the curved shape when the catheter is curved to one side of the leaf spring and the curved shape when the catheter is curved to the other side of the leaf spring are different. This allows the distal part of the catheter of the present invention to be easily delivered to a desired location in a blood vessel or the heart. Different curved shape means that the shape when curved on one side is different from the shape when curved on the other side, including the size of the circle along which the curved shape is aligned, in other words, the radius of the circle is different. According to the present invention, the difference in curve shape, especially in size, between the curved shape on one side and the curved shape on the other side can be made larger, so that the curves of various shapes of blood vessels can be accommodated.

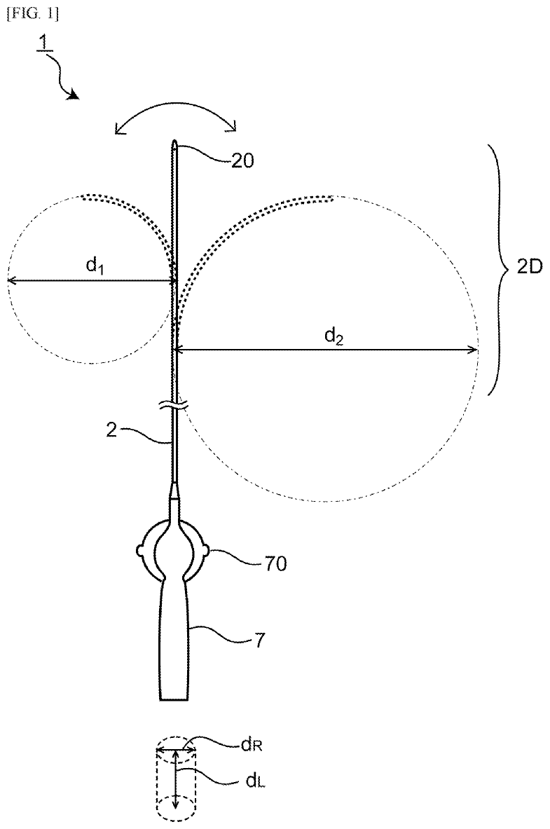

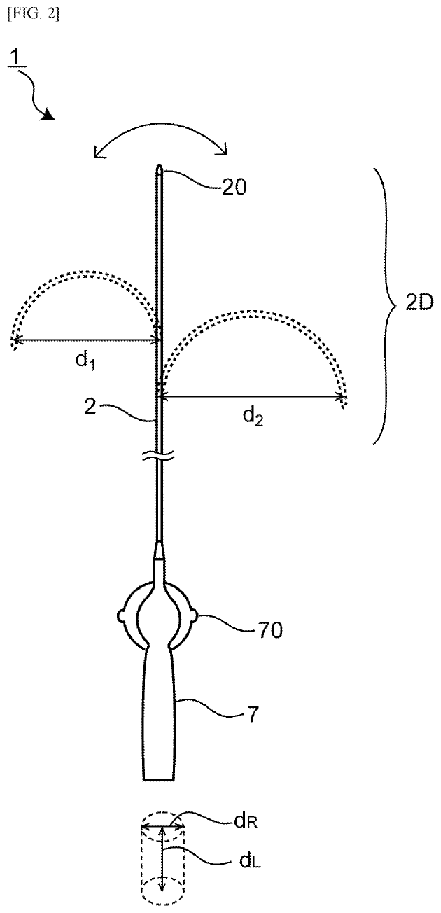

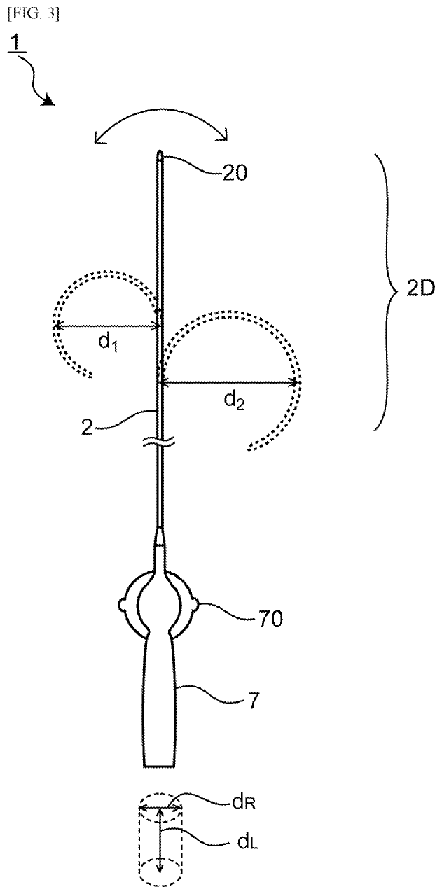

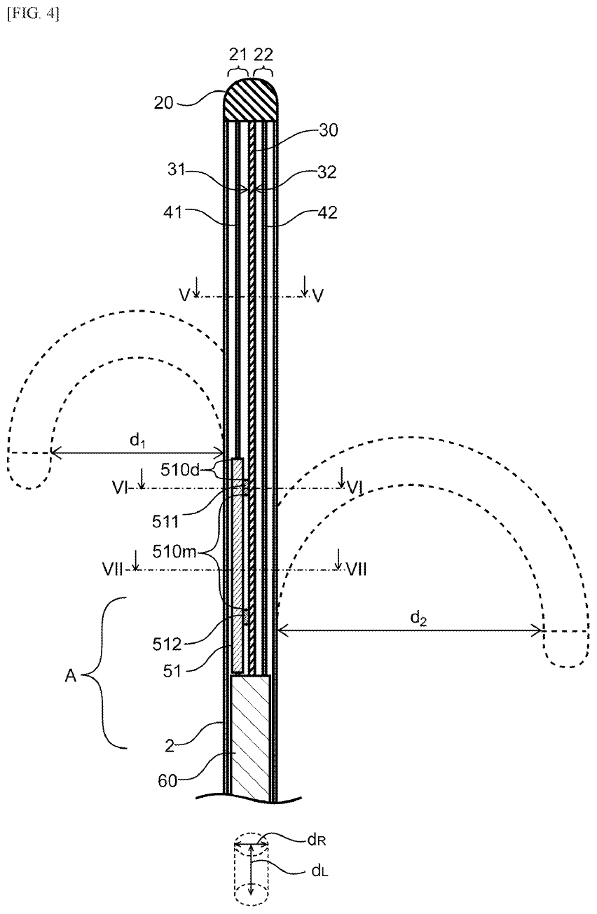

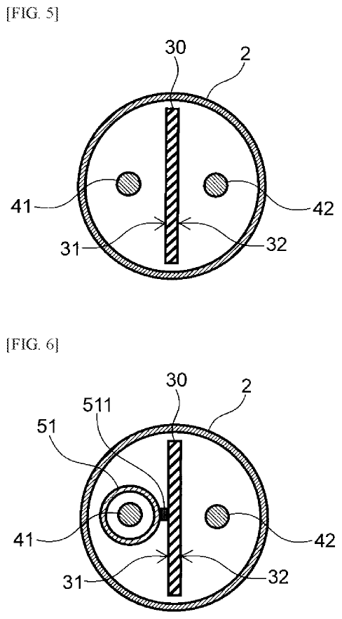

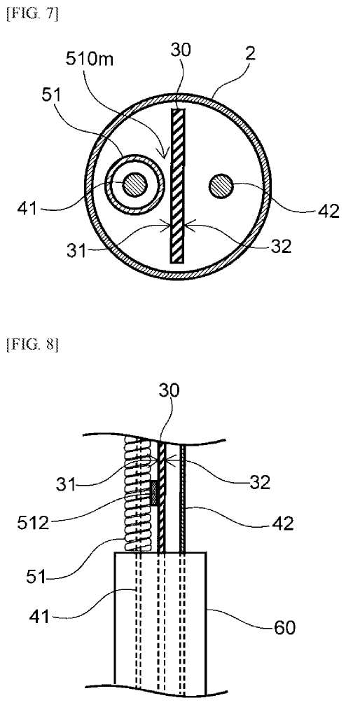

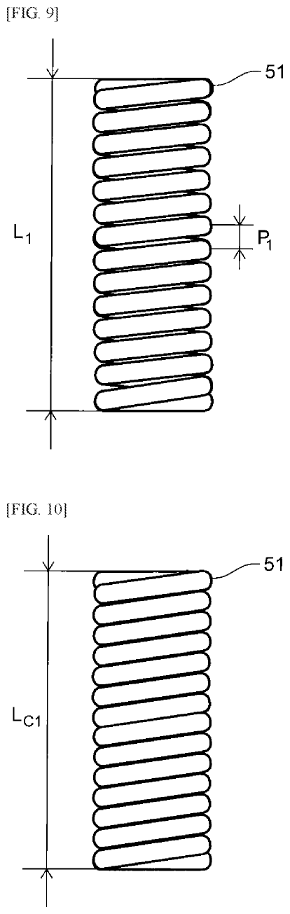

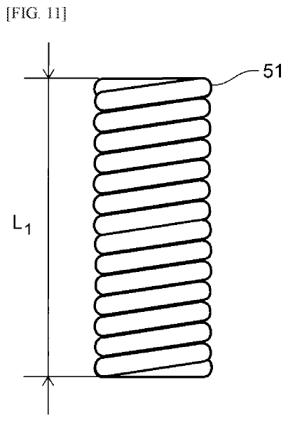

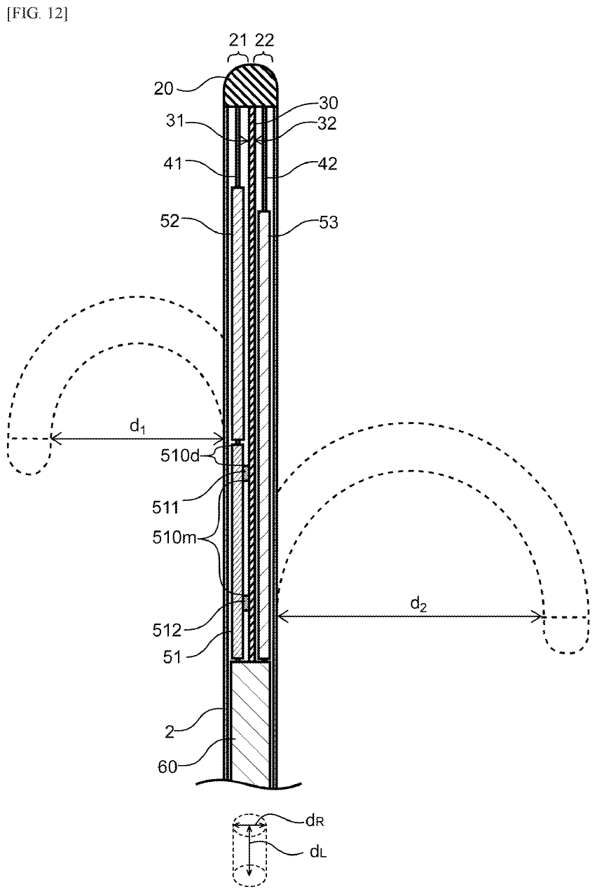

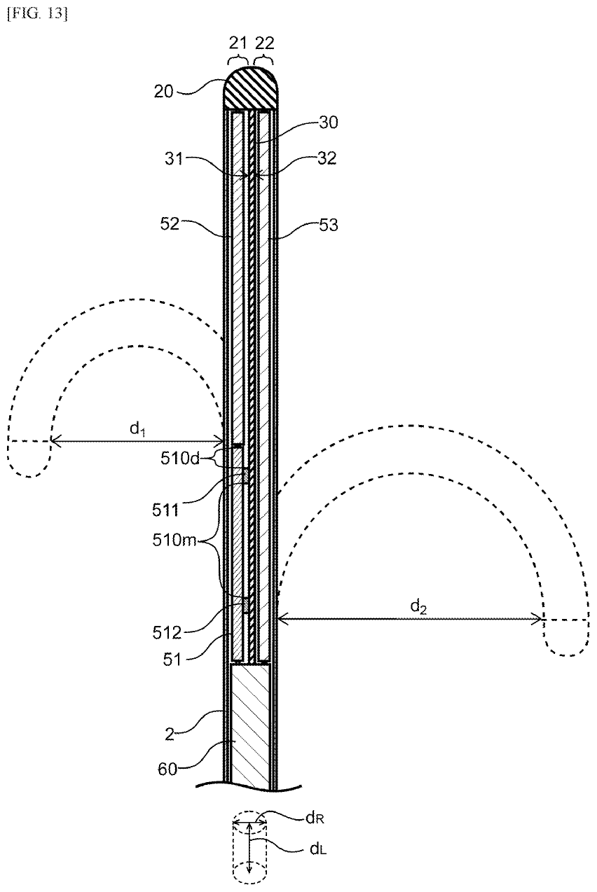

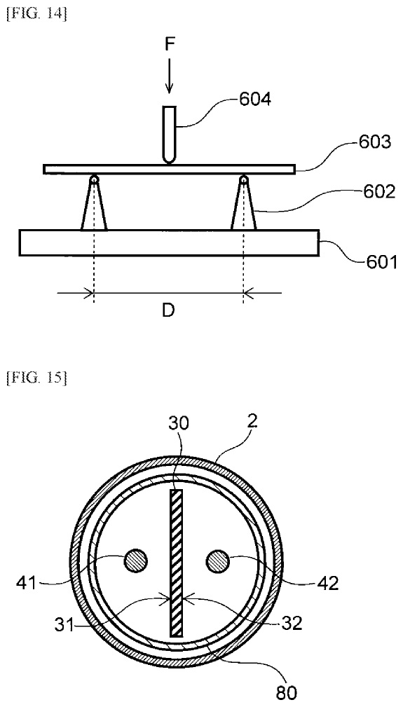

Hereinafter, a catheter according to embodiments of the present invention will be described referring to FIG. 1 to FIG. 15. FIG. 1 to FIG. 3 are plan views of a catheter according to an embodiment of the present invention, and the dotted lines represent the appearance when the distal part of the catheter is curved on one side and the other side of the leaf spring in the radial direction of the shaft. The dotted lines in FIG. 2 represent the situation when the distal part of the catheter is curved further than shown in FIG. 1. The dotted lines in FIG. 3 represent the situation when the distal part of the catheter is curved further than shown in FIG. 2. FIG. 4 is a cross-sectional view of the distal part of the catheter shown in FIG. 2 in the longitudinal direction, and the dotted lines represent the situation when the distal part of the catheter is curved on one side and the other side of the leaf spring in the radial direction of the shaft. FIG. 5 to FIG. 7 are a V-V cross-sectional view, a VI-VI cross sectional view, and VII-VII cross-sectional vies of the distal part of the catheter shown in FIG. 4, respectively. FIG. 8 is a side view of a first wire, second wire, leaf spring, supporting member, and first coil located in the lumen of the shaft in part A of the distal part of the catheter shown in FIG. 4. FIG. 9 is a side view of a first coil according to an embodiment of the present invention in its natural state, and FIG. 10 is a side view of the first coil shown in FIG. 9 at maximum compression. FIG. 11 is a side view of a first coil according to another embodiment of the present invention in its natural state. FIG. 12 is a cross-sectional view of a distal part of a catheter according to another embodiment of the present invention in the longitudinal direction, and the dotted lines represent the situation when the distal part of the catheter is curved on one side and the other side of the leaf spring in the radial direction of the shaft. FIG. 13 is a cross-sectional view of a distal part of a catheter according to still another embodiment of the present invention in the longitudinal direction, and the dotted lines represent the situation when the distal part of the catheter is curved on one side and the other side of the leaf spring in the radial direction of the shaft. FIG. 14 shows a stiffness measurement method. FIG. 15 shows a different example of the V-V cross-sectional view of the distal part of the catheter shown in FIG. 4.

In the present invention, a proximal side refers to the user's hand side in the direction of shaft extension, and a distal side refers to the opposite side of the proximal side, i.e., the side of the person to be treated. The direction of shaft extension is referred to as a longitudinal direction d_{L}. The radial direction perpendicular to the direction of shaft extension is referred to as a radial direction d_{R}. In FIGS. 1 to 4, 8, 12, and 13, the lower side of the figure is the proximal side and the upper side of the figure is the distal side. In FIGS. 1 to 4, 8, 12, and 13, the left side of the figure is one side of the leaf spring in the radial direction d_{R} of the shaft, and the right side of the figure is the other side of the leaf spring in the radial direction d_{R} of the shaft.

As shown in FIG. 1 to FIG. 3, a catheter 1 has a shaft 2 having a distal end, a proximal end, and a lumen extending in the longitudinal direction d_{L}. A tip part 20 is preferably disposed at the distal end of the shaft 2, and a handle 7 is preferably disposed at a proximal end part of the shaft 2.

The distal end of the shaft 2 is inserted into the body, and delivered to a treatment site. For this reason, preferably, it is flexible, and metal or resin can be used as the material. Since it is inserted into the body, it is preferable to use biocompatible materials. On the surface of the shaft 2, devices for treatment, such as electrodes and sensors, can be placed. By providing electrodes on the surface of the shaft 2, it can be used as an electrode catheter to measure cardiac potential or an ablation catheter to cauterize tissue.

Internal structures for curving the catheter, or devices and their internal structures for treatment, such as sensors and conductors, for example may be placed in the lumen of the shaft 2. The lumen of the shaft 2 may be a single lumen or partially multiple lumens. The structures for curvature of the present invention are arranged in a single lumen. For example, to bend the shaft distal part 2D of the catheter 1, the part to be curved can be a single lumen, and proximal to the single lumen there may be multiple lumens. The lumen may have a double structure. The length in the longitudinal direction d_{L}, outer diameter, thickness, etc. of the shaft can be selected to be the appropriate size for the treatment.

The tip part 20 is preferably disposed at the distal end of the shaft 2. The tip part 20 may be a member different from or same as the shaft 2. When the tip part 20 is a member different from the shaft 2, the tip part 20 may be provided with a part that is inserted into the lumen of the shaft 2 or a part that projects distally from the distal end of the shaft. When the tip part 20 is a member integrated with the shaft 2, the distal end part of the shaft 2 may be formed by sealing the opening at the distal end of the shaft 2 by heat fusion or other means to form the tip part 20.

The handle 7 is preferably disposed at the proximal side of the shaft 2. The proximal end of the shaft 2 is preferably fixed to the inside of the handle 7. A conductor or operating wire extending from the lumen of the shaft 2 is disposed within the handle 7. To facilitate manipulation of the operating wire, the handle 7 may include a wire manipulator 70. By fixing the proximal end of the operating wire to the wire manipulator 70, and operating the wire manipulator 70 to pull the wire, the distal end of the catheter 1 can be bent.

As shown in FIG. 4 to FIG. 7, the catheter 1 has, in the lumen of the shaft 2, a first wire 41 and a second wire 42 each having a distal end and a proximal end and extending in the lumen of the shaft 2 (for example, extending from the tip part 20 to the handle 7), whose distal end is fixed to the distal end part of the shaft 2, for example, to the tip part 20, whose proximal end is placed at a proximal end part of the shaft 2, for example, in the handle 7; a leaf spring 30 having a distal end and a proximal end and being disposed in the lumen of the shaft 2 so as to separate the lumen into a first part 21 where the first wire 41 is disposed and a second part 22 where the second wire 42 is disposed in the longitudinal direction d_{L}; and a supporting member 60, to which the proximal end of the leaf spring 30 is fixed, having a distal end and a proximal end, extending in the longitudinal direction d_{L}, having a lumen in which the first wire 41 and the second wire 42 are disposed, and being disposed proximal to the leaf spring 30.

The first wire 41 and the second wire 42 are operating wires used to control the curvature of the shaft distal part 2D of the catheter 1. The first wire 41 and the second wire 42 are disposed in the lumen of the shaft 2, whose distal end is preferably fixed to the tip part 20 and whose proximal end is preferably fixed to the handle 7. As the first wire 41 and the second wire 42, metal wires such as stainless steel or wires formed from synthetic resins such as fluoroplastics can be used. The first wire 41 and the second wire 42 may each be a single wire or may have a structure consisting of multiple wires.

The leaf spring 30 is a member that defines the direction of curvature of the catheter 1 and is placed in the lumen of the shaft 2 so as to separate the lumen of the shaft 2 into a first part 21 where the first wire 41 is disposed and a second part 22 where the second wire 42 is disposed in the longitudinal direction d_{L}. The proximal end of the leaf spring 30 is fixed to the supporting member 60. The distal end of the leaf spring 30 is preferably fixed to the distal end part of the shaft 2. In the case where the tip part 20 is provided at the distal end of the shaft 2, the distal end of the leaf spring 30 is preferably fixed to the tip part 20. The distal end of the leaf spring 30 may not be fixed. The distal and proximal ends of the leaf spring 30 may be fixed by not fixing the ends directly, but by fixing the vicinity of the ends. The method of fixing the distal and proximal ends of the leaf spring 30 is not limited, and may include, for example, blazing such as solder, welding, adhesive bonding, and connection by crimping. When the tip part 20 and the supporting member 60 are metal, they are preferably fixed by laser welding.

The leaf spring 30 has a distal end and a proximal end, and is shaped to extend in the longitudinal direction d_{L}. The leaf spring 30 is preferably placed along the longitudinal axis of the shaft 2. This allows the lumen of the shaft 2 to be separated by the leaf spring 30 into the first part 21 on one side including the longitudinal axis of the shaft 2 and the second part 22 on the other side. The first wire 41 is disposed in the first part 21, which is one part of the separated lumen of the shaft 2, and the second fire 42 is disposed in the second part 22, which is the other part. The surface of the leaf spring 30 on the side where the first wire 41 is disposed is referred to as a first surface 31, and the surface of the leaf spring 30 on the side where the second wire 42 is disposed is referred to as a second surface 32. The first surface 31 of the leaf spring 30 can be referred to as one side and the second surface 32 can be referred to as the other side.

The leaf spring 30 is a spring using a plate material, and the materials that make up the leaf spring 30 includes metals such as stainless steel, titanium, carbon steel, nickel-titanium alloys, cobalt-chrome alloys, tungsten alloys. Alternatively, the materials that make up the leaf spring 30 may include synthetic resin such as aromatic polyetherketone resin (e.g., PEEK), polycarbonate resin, and fiber-reinforced resin. Alternatively, the leaf spring 30 may be made of natural rubber or synthetic rubber such as butadiene rubber, isoprene rubber, styrene butadiene rubber, ethylene propylene rubber, acrylic rubber, and silicone rubber. Of these, the material of the leaf spring 30 is preferably stainless steel.

The supporting member 60 is disposed proximal to the leaf spring 30 in the lumen of the shaft 2, and the first wire 41 and the second wire 42 are disposed in the lumen of the supporting member 60. The proximal end of the leaf spring 30 is fixed to the distal end of the supporting member 60. The proximal end of the supporting member 60 may extend to the proximal end of the shaft 2, or may be located in the middle of the shaft 2. The supporting member 60 may switch to a different member, such as a tube, in the middle of the shaft 2.

The supporting member 60 may be a proximal tube having a cylindrical shape. When the supporting member 60 is a proximal tube, the proximal tube can receive the proximal end of the leaf spring 30 and a part of the leaf spring 30 can be placed in the lumen of the proximal tube. This can strengthen the fixation between the leaf spring 30 and the supporting member 60.

The support member 60, like the shaft 2, preferably has flexibility, and can be made of metal or resin. In particular, it is preferably a coil wound with metal wire. Since the internal structure of the catheter 1 of the present invention switches at the distal end of the supporting member 60, the size, flexibility, and materials of the supporting member 60 is preferably selected so that the change in stiffness of the catheter 1 between the distal and proximal side with respect to the distal end of the supporting member 60 does not become significant.

The catheter 1 of the present invention can be curved in the section from where the leaf spring 30 is exposed from the distal end of the supporting member 60 to where the leaf spring 30 is fixed to the distal end part of the shaft 2, for example, the tip part 20. Therefore, the length of the curved portion of the catheter 1 can be appropriately set according to the length of the leaf spring 30, the position at which the leaf spring 30 is fixed to the tip part 20 and the supporting member 60. Preferably, the supporting member 60 is not easily deformed by the curvature of the leaf spring 30.

As shown in FIG. 4, the catheter 1 has a first coil 51 having a lumen in which the first wire 41 is disposed, and being disposed distal to the distal end of the supporting member 60. The first coil 51 is located on the proximal end side of the leaf spring 30, and fixed to the proximal side of the leaf spring 30 at two or more locations. Being located on the proximal end side of the leaf spring 30 means that the first coil 51 is located closer to the proximal end of the leaf spring, and the proximal end of the first coil 51 is located adjacent or closer to the distal end of the supporting member 60. Since the first coil 51 is a member that controls the curvature of the leaf spring 30 to the side of the first surface 31, if the first coil 51 is fixed to the distal side of the leaf spring 30, the leaf spring 30 cannot bend toward the side of the first surface 31. The first coil 51 has a first fixed portion 511 where the first coil 51 and the leaf spring 30 are fixed, a second fixed portion 512 located proximal to the first fixed portion 511, and an intermediate unfixed portion 510m where the first coil 51 and the leaf spring 30 are not fixed located between the first fixed portion 511 and the second fixed portion 512.

The first coil 51 can be made of metal wire such as stainless steel and nickel-titanium alloys, or synthetic resin wire such as aromatic polyetherketone resin (e.g., PEEK) and polycarbonate resin. The cross-sectional shape of the wire forming the first coil 51 can be circular, rectangular, or a combination thereof. Of these, the first coil 51 is preferably a metal coil made of stainless steel wire with a circular cross-sectional shape. The wire diameter, coil diameter, and length of the first coil 51 can be appropriately selected as needed.

The first coil 51 and the leaf spring 30 are preferably fixed by welding, soldering, bonding, or crimping. Of these, fixing by welding is preferred. When fixed by welding, the first fixed portion 511 and the second fixed part 512 can be formed without materials such as solder and adhesive.

The first coil 51 may be fixed directly to the leaf spring 30, or may be fixed to the leaf spring 30 by the proximal end of the first coil 51 contacting the supporting member 60, or the first coil 51 and the leaf spring 30 may be fixed indirectly by the first coil 51 being fixed to the supporting member 60 by welding, soldering, bonding, or crimping. The second fixed portion 512 may be fixed by the proximal end of the first coil 51 contacting the supporting member 60, or the first coil 51 may be fixed to the supporting member 60 by welding, soldering, bonding, or crimping, thereby indirectly fixing the first coil 51 and the leaf spring 30 to form the second fixed portion 512.

The location in the longitudinal direction d_{L} of the first fixed portion 511 and the second fixed portion 512 is not limited, and may be located anywhere on the surface of the first coil 51. For example, the first fixed portion 511 may be located at the distal end of the first coil 51, and the second fixed portion 512 may be located at the proximal end of the first coil 51. Although not shown in the figures, additional fixed portions other than the first fixed portion 511 and second fixed portion 512 may be provided. The total number of fixed portions is at least two, and more than two fixed portions may be provided. By providing at least two fixed portions, the first coil 51 can be fixed to the leaf spring 30 so that the leaf spring 30 proximal to the most distal fixed portion does not bend toward the side of the first surface 31. To prevent the difference in stiffness between the portion of the shaft 2 where the first coil 51 is located and the rest of the shaft 2 from becoming too large, the number of the fixed portions is preferably two.

The length in the longitudinal direction d_{L} of the fixed portions of the first coil 51 and the leaf spring 30 including the first fixed portion 511 and the second fixed portion 512 can be appropriately determined, however, the shorter the length in the longitudinal direction d_{L} of the fixed portions is, the more preferable, and for example, about the length of three winds of the wire forming the coil is preferable as shown in FIG. 8. If the length in the longitudinal direction d_{L} of the fixed portion is long, the catheter 1 may lose flexibility when it bends toward the side of the second surface 32.

As shown in FIG. 9 and FIG. 10, the first coil 51 has a total length L₁ in its natural state and a total length L_{C1} at maximum compression. The ratio L_{C1}/L₁ is 0.9 or more. In addition, the first coil 51 is preferably uncompressed as shown in FIG. 11. Note that an uncompressed coil is a closely wound coil, that is, a coil that is not compressed in the longitudinal direction d_{L}, and the ratio L_{C1}/L₁ of the total length L_{C1} of the coil at maximum compression to the total length L₁ of the coil in its natural state is 1. However, a coil with the ratio L_{C1}/L₁ of 0.9 or more, or 0.95 or more is substantially uncompressed, and included in the uncompressed coil in the present invention. To achieve a high degree of incompressibility of the first coil 51, L_{C1}/L₁ is preferably 0.95 or more.

With the configuration of the present invention, the shaft distal part 2D of the catheter 1 can be curved on both one side and the other side, each with a different curved shape. In particular, each curvature diameter can be different, and the difference in curvature diameter can be precisely controlled. When the first wire 41 disposed in the first part 21 of the lumen of the shaft 2, in other words, the first wire 41 disposed on the side of the first surface 31 of the leaf spring 30 is pulled, the leaf spring 30 is curved toward the side of the first surface 31, and the shaft distal part 2D of the catheter 1 is accordingly curved toward the side of the first surface 31. Since the first coil 51 is fixed to the proximal side of the leaf spring 30 at two or more locations and the first coil 51 is uncompressed, the first coil cannot bend toward the side opposite to the leaf spring 30, that is, the side of the first surface 31. This is because the first coil 51 functions and behaves as a rigid tube, because the uncompressed coil is fixed at two points, and the first coil 51 cannot be compressed between those two points against curvature to the one side. Therefore, only the portion of the leaf spring 30 between the portion fixed to the tip part 20 and the most distal fixed portion to the first coil 51 can be curved to the one side.

If the leaf spring 30 and the first coil 51 are fixed at only one point, then the first coil 51 can bend to the side of the first surface 31 along with the curvature of the leaf spring 30. In addition, if the first coil 51 is not an uncompressed coil, then the coil can be stretched to both the one side and the other side, therefore, can be curved to the one side. Accordingly, if the first coil 51 lacks either fixation with the leaf spring 30 at two points or incompressibility, the leaf spring 30 can be curved in the range from the distal side to the proximal side toward the side of the first surface 31.

On the other hand, when the second wire 42 disposed in the second part 22 of the lumen of the shaft 2 of the catheter 1, in other words, the second wire 42 disposed on the side of the second surface 32 of the leaf spring 30 is pulled, the leaf spring 30 is curved toward the side of the second surface 32, and the shaft distal part 2D of the catheter 1 is accordingly curved toward the side of the second surface 32. The interval of winds of the coil wire at the unfixed portion on the one side of the first coil 51 disposed on the side of the first surface 31 of the leaf spring 30 becomes widened, and the first coil 51 can be bend toward the side of the second surface 32 following the curvature of the leaf spring 30 toward the side of the second surface 32. Thus, all ranges of the leaf spring 30 from the portion fixed to the tip part 20 to the distal end of the supporting member 60 can be curved toward the side of the second surface 32.

The portion where the first coil 51 and the leaf spring 30 are fixed is preferably located on the side of the first coil 51 facing the first surface 31 of the leaf spring 30. Thereby, curvature of the first coil 51 toward the side of the leaf spring 30, i.e., the side of the second surface 32 does not prevented.

The intermediate unfixed portion 510m including no fixed portion between the first fixed portion 511 and the second fixed portion 512 of the first coil 51 preferably has a length in the longitudinal direction d_{L} in its natural state of 50% or longer of the total length L₁ of the first coil 51 in its natural state. In the case where more than one intermediate unfixed portion 510m is provided, the length of the longest intermediate unfixed portion 510m in its natural state in the longitudinal direction d_{L} is preferably 50% or longer of the total length L₁ of the first coil 51 in its natural state. By keeping the length of the intermediate unfixed portion 510m long, the first coil 51 can be bend toward the side of the second surface 32 without hindrance while preventing the first coil 51 from bending toward the side of the first surface 31. The length of the longest intermediate unfixed portion 510m of the first coil 51 in its natural state in the longitudinal direction d_{L} may be 30% or longer of the total length L₁ of the first coil 51 in its natural state, or may be 20% or longer. Even in such a range, the first coil 51 can be bend toward the side of the second surface 32 without hindrance while preventing the first coil 51 from bending toward the side of the first surface 31. In particular, when the fixed portion distal to the longest intermediate unfixed portion 510m is close to the distal side of the first coil 51, even if the length of the longest intermediate unfixed portion 510m of the first coil 51 in its natural state in the longitudinal direction d_{L} is shorter than 50% of the total length L₁ of the first coil 51 in its natural state, the curvature of the first coil 51 toward the side of the first surface 31 can be effectively prevented.

By controlling the position of the fixed portion of the first coil 51, the shape of curvature of the distal bent part of the catheter 1 can be controlled. This is because the curvature to the side of the first surface 31 is formed by the portion of the first wire 41 between the fixed portion at the distal end part of the shaft 2 and the most distal fixed part of the first coil 51, and the curvature to the side of the second surface 32 is formed by the portion of the second wire 42 between the fixed portion at the distal end part of the shaft 2 and the distal end of the supporting member 60.

Preferably, the first coil 51 does not have a fixed portion where the first coil 51 and the leaf spring 30 are fixed between the distal end of the first coil 51 and the first fixed portion 511. By providing such a distal unfixed portion 510d extending between the distal end of the first coil 51 and the most distal fixed portion, extreme changes can be prevented in the shape of curvature between the bendable portion distal to the first coil 51 and the portion that is located proximal to the most distal fixed portion of the first coil 51 and does not bend toward the side of the first surface 31. For example, if the most distal fixed portion of the first coil 51 is located at the distal end of the first coil 51, the catheter 1 may break at the distal end of the first coil 51.

As shown in FIG. 12, the catheter 1 may further have a second coil 52. The second coil 52 has a lumen in which the first wire 41 is disposed, and is disposed distal to the first coil 51 and in the first part 21 that is formed by the leaf spring 30 separating the lumen of the shaft 2. The second coil 52 may be in contact with any part such as leaf spring 30 in the lumen of the shaft 2, however, it is preferred that the second coil 52 is not fixed to any part.

As shown in FIG. 12, the catheter 1 may further have a third coil 53. The third coil 53 has a lumen in which the second wire 42 is disposed, and is disposed in the second part 22 that is formed by the leaf spring 30 separating the lumen of the shaft 2. The third coil 53 may be in contact with any part such as leaf spring 30 in the lumen of the shaft 2, however, it is preferred that the third coil 53 is not fixed to any part.

Although not shown in the figures, similar to FIG. 9 and FIG. 10 for the first coil 51, the second coil 52 has a total length L₂ in its natural state and a total length L_{C2} at maximum compression. The ratio L_{C2}/L₂ is preferably less than 0.9, and preferably less than L_{C1}/L₁. Similarly, the third coil 53 has a total length L₃ in its natural state and a total length L_{C3} at maximum compression. The ratio L_{C3}/L₃ is preferably less than 0.9, and preferably less than L_{C1}/L₁. If the ratio L_{C1}/L₁ of the total length L_{C1} at maximum compression to the total length L₁ in its natural state of the first coil 51 is the same as or smaller than the ratio L_{C2}/L₂ of the second coil 52 and the ratio L_{C3}/L₃ of the third coil 53, the first coil 51 is equally or more easily deformed than the second coil 52 and the third coil 53, making it difficult to control the curved shape of the catheter 1.

The length of the first coil 51, the second coil 52, and the third coil 53 can be appropriately selected according to the shape of the required curvature of the catheter 1. The first wire 41 and the second wire 42 may be partially exposed between the tip part 20 and the supporting member 60 as shown in FIG. 12, or may be placed entirely in the lumen of the first coil 51, the second coil 52, and the third coil 53 as shown in FIG. 13. For example, the first coil 51 and the third coil 53 may be placed so as to be in contact with the supporting member 60. Alternatively, the first coil 51 and the third coil 53 may be placed at a distance from the supporting member 60 so that the first wire 41 and the second wire 42 are exposed. This is the same with the relationship between the tip part 20 and the second coil 52 and the third coil 53. Similarly, the first coil 51 and the second coil 52 may be placed so as to be in contact with each other, or placed at a distance from each other.

As shown in FIG. 9, the first coil 51, the second coil 52, and the third coil 53 consist of helically wound coil wire. The pitch interval of the first coil 51, the second coil 52, and the third coil 53 can be appropriately determined. The pitch interval of a coil is the distance between the center point of the adjacent winds of the coil wire forming the coil. The pitch interval can be measured as the pitch interval P₁ of the first coil 51 shown in FIG. 9. When the pitch interval is larger than the thickness of the coil wire, there is a gap between the winds of the coil wire and the coil can be compressed. In this case, the ratio L_{C}/L of the total length L_{C} at maximum compression to the total length L in its natural state becomes smaller than 1. Accordingly, L_{C}/L of the present invention approximates coil diameter/pitch interval. When L_{C1}/L₁ of the first coil 51 is 0.9 or more, the coil diameter/pitch interval of the first coil 51 is 0.9 or more.

When the catheter 1 is provided with the second coil 52 and the third coil 53, the coil wire diameter and the coil diameter of the second coil 52 and the third coil 53 can be appropriately determined according to the shape of the required curvature of the catheter 1. The materials for the second coil 52 and the third coil 53 are the same as those listed as materials for the first coil, and metals and resins can be used. Not only the pitch interval, but also the coil wire diameter, coil diameter, and material of the coil can control the total length L of the coil in its natural state and the total length L_{C} at maximum compression.

When the catheter 1 is provided with the second coil 52 and the third coil 53, the pitch interval P₁ of the first coil 51 is preferably shorter than the pitch interval of the second coil 52 and the pitch interval of the third coil 53. By setting the pitch interval in this way, the total length L of the coil in its natural state and the total length L_{C} of the coil at maximum compression can be properly adjusted, so that L_{C2}/L₂ and z L_{C3}/L₃ can be made smaller than L_{C1}/L₁.

Among other things, when the catheter 1 is provided with the second coil 52, the pitch interval P₁ of the first coil 51 is preferably shorter than the pitch interval of the second coil 52. In addition, in this case, the coil wire diameter and the coil diameter of the first coil 51 is preferably the same as the coil wire diameter and the coil diameter of the second coil 52. The coil wire diameter is a diameter of the wire forming the coil, and the coil diameter is the diameter of the coil. This allows the size of the internal structure of the shaft 2 to be balanced, and also allows the curved shape toward the side of the second surface 32 to be made closer to a circular shape because the stiffness of the first coil 51 and the second coil 52 when bending toward the side of the second surface 32 can be made similar.

The pitch interval of the second coil 52 may be the same as or different from the pitch interval of the third coil 53. The pitch interval of the second coil 52 and the pitch interval of the third coil 53 only need to be adjusted such that the coils do not compress completely when curved to the maximum extent. Because of the presence of the first coil 51, the curvature diameter of the bend of the catheter 1 toward the side of the first surface 31 is smaller than the curvature diameter of the bend toward the side of the second surface 32, and the second coil 52 is shorter than the third coil 53. Since the second coil 52 is shorter than the third coil 53, the second coil 52 is more likely to be fully compressed, and in this regard, it is preferred to set the pitch interval of the second coil 52 wider. The coil wire diameter and coil diameter of the second coil 52 may be the same as or different from the coil wire diameter and coil diameter of the third coil 53, respectively, however, they are preferably the same. The pitch interval of the second coil 52 is preferably different from the pitch interval of the third coil 53, thereby the shape of curvature toward the side of the first surface 31 can be made more different from the shape of curvature toward the side of the second surface 32 of the catheter 1. The coil wire diameter and coil diameter of the first coil 51 may be the same as or different from the ones of the second coil 52 and the third coil 53, respectively.

The bending stiffness of the first coil 51 is preferably greater than the bending stiffness of the second coil 52, and the difference between the bending stiffness of the first coil 51 and the bending stiffness of the second coil 52 is preferably 50% or less. This allows for a smooth curvature in the shape of the bend when the shaft distal part 2D bends toward the side of the first surface 31 of the leaf spring 30. Methods of adjusting the stiffness include selecting the type and amount of wire material comprising each coil, adjusting the inner and outer diameter of each coil, and adjusting the pitch interval of each coil.

The stiffness can be obtained by three-point bending test as shown in FIG. 14. The three-point bending test was performed in accordance with JIS K7171. A stiffness measurement sample 603 is placed on two supporting points 602 that are placed on a support stand 601 such that they are separated by a distance D between the supporting points, and load F is measured when an indenter 604 that is placed at the center of the distance D between the supporting points is moved a certain distance vertically, and the load F is determined to be the stiffness of the stiffness measurement sample 603.

As shown in FIG. 1 to FIG. 4, the catheter 1 having the above configuration has a curvature shape when the shaft distal part 2D is curved toward the side of the first surface 31 of the leaf spring 30 different from a curvature shape when the shaft distal part 2D is curved toward the side of the second surface 32 of the leaf spring 30 thanks to the first coil 51. Preferably, this allows a curvature diameter d₁ when the shaft distal part 2D is curved toward the side of the first surface 31 of the leaf spring 30 smaller than a curvature diameter d₂ when the shaft distal part 2D is curved toward the side of the second surface 32 of the leaf spring 30. Note that the curvature diameter d₁ is a diameter of a circumscribed circle of the outside of the shaft distal part 2D on the side of the first wire 41 when curved toward the side of the first surface 31 of the leaf spring 30, and when the curvature of the shaft distal part 2D is a part of a circular arc, it is the diameter of the circle. The curvature diameter d₂ is a diameter of a circumscribed circle of the outside of the shaft distal part 2D on the side of the second wire 42 when curved toward the side of the second surface 32 of the leaf spring 30, and when the curvature of the shaft distal part 2D is a part of a circular arc, it is the diameter of the circle. Depending on the condition of the curvature, the shape of the curvature may be a shape of a combination of straight and curved lines instead of a circumscribed circle or a circular arc. In any case, the catheter 1 of the present invention can make the curvature shape at the side of the first surface 31 different from the curvature shape at the side of the second surface 32 thanks to the first coil 51.

As shown in FIG. 15, preferably, the catheter 1 further has a protection tube 80 having a lumen and being disposed in the lumen of the shaft 2, and the leaf spring 30, the first coil 51, the second coil 52, and the third coil 53 are placed in the lumen of the protection tube 80. To form the protection tube 80, similar materials for forming the shaft 2 can be used. The protection tube 80 can protect the leaf spring 30, the first coil 51, the second coil 52, and the third coil 53, and further the first wire 41 and the second wire 42. The protection tube 80 may be in contact with any part, and, preferably fixed to the supporting member 60. This can prevent the coils from moving in the shaft 2 thanks to the protection tube 80.

The present application claims priority based on Japanese Patent Application No. 2020-97338 filed on Jun. 4, 2020. All the contents described in Japanese Patent Application No. 2020-97338 filed on Jun. 4, 2020 are incorporated herein by reference.

### DESCRIPTION OF REFERENCE SIGNS

1: catheter
2: shaft
2D: shaft distal part
7: handle
20: tip part
21: first part
22: second part
30: leaf spring
31: first surface of the leaf spring
32: second surface of the leaf spring
41: first wire
42: second wire
51: first coil
52: second coil
53: third coil
60: supporting member
70: wire manipulator
80: protection tube
510d: distal unfixed portion
510m: intermediate unfixed portion
511: first fixed portion
512: second fixed portion
601: support stand
602: supporting point
603: stiffness measurement sample
604: indenter
d₁: curvature diameter when curved toward the side of the first surface of the leaf spring
d₂: curvature diameter when curved toward the side of the second surface of the leaf spring
d_{L}: longitudinal direction
d_{R}: radial direction
D: distance between supporting points
F: load
L₁: total length of the first coil in its natural state
L_{C1}: total length of the first coil at maximum compression
P₁: pitch interval of the first coil

1. A catheter, comprising:
a shaft having a distal end, a proximal end, and a lumen extending in a longitudinal direction;
a first wire having a distal end and a proximal end and extending in the lumen of the shaft, the distal end being fixed to a distal end part of the shaft, and the proximal end being disposed at a proximal end part of the shaft;
a second wire having a distal end and a proximal end and extending in the lumen of the shaft, the distal end being fixed to a distal end part of the shaft, and the proximal end being disposed at a proximal end part of the shaft;
a leaf spring disposed in the lumen of the shaft so as to separate the lumen of the shaft into a first part where the first wire is disposed and a second part where the second wire is disposed in the longitudinal direction;
a supporting member, to which the proximal end of the leaf spring is fixed, extending in the longitudinal direction, having a lumen in which the first wire and the second wire are disposed, and being disposed proximal to the leaf spring; and
a first coil having a lumen in which the first wire is disposed and being disposed in the first part and distal to a distal end of the supporting member, wherein
the first coil is fixed to a proximal end side of the leaf spring at two or more locations, which include a first fixed portion and a second fixed portion so that the first coil has at least one unfixed portion including an intermediate unfixed portion, which is not fixed to the leaf spring and is located between the first fixed portion and the second fixed portion,
the second fixed portion is located proximal to the first fixed portion, and
the first coil has a total length L₁ in its natural state and a total length L_{C1} at maximum compression, and a ratio L_{C1}/L₁ is 0.9 or more.

2. The catheter according to claim 1, wherein the supporting member is a tubular member.

3. The catheter according to claim 1, wherein the first coil is a closely wound coil.

4. The catheter according to claim 1, wherein an outer surface of the first coil is fixed to a surface of the leaf spring at the first fixed portion and the second fixed portion, the surface of the leaf spring defining a part of the first part of the lumen of the shaft.

5. The catheter according to claim 1, wherein an unfixed portion having a longest length in the longitudinal direction among the at least one unfixed portion has a length in its natural state in the longitudinal direction of 50% or longer of the total length L₁ of the first coil in its natural state.

6. The catheter according to claim 1, wherein the first coil is disposed in the first part of the lumen of the shaft so that the first coil has a distal unfixed portion, which does not include a fixed portion where the first coil and the leaf spring is fixed, between a distal end of the first coil and the first fixed portion.

7. The catheter according to claim 1, further comprising a second coil having a lumen in which the first wire is disposed and being disposed in the first part and distal to the first coil.

8. The catheter according to claim 7, wherein the second coil has a total length L₂ in its natural state and a total length L_{C2} at maximum compression, and the ratio L_{C2}/L₂ is less than 0.9.

9. The catheter according to claim 7, wherein a bending stiffness of the first coil is greater than a bending stiffness of the second coil, and the difference between the bending stiffness of the first coil and the bending stiffness of the second coil is 50% or less.

10. The catheter according to claim 7, wherein
the first coil is formed of a helically wound first coil wire,
the second coil is formed of a helically wound second coil wire, and
a pitch interval of the first coil is shorter than a pitch interval of the second coil.

11. The catheter according to claim 10, wherein a coil wire diameter of the first coil is the same as a coil wire diameter of the second coil, and a coil diameter of the first coil is the same as a coil diameter of the second coil.

12. The catheter according to claim 1, further comprising a third coil having a lumen, wherein the third coil is disposed in the second part of the lumen of the shaft and the second wire is disposed in the lumen of the third coil.

13. The catheter according to claim 12, wherein the third coil has a total length L₃ in its natural state and a total length L_{C3} at maximum compression, and the ratio L_{C3}/L₃ is less than 0.9.

14. The catheter according to claim 1, further comprising:
a second coil having a lumen,
a third coil having a lumen, and
a protection tube having a lumen, wherein the second coil is disposed in the first part of the lumen of the shaft, and the first wire is disposed in the lumen of the first coil and the lumen of the second coil so that the second coil is located at a distal side of the first coil,
the third coil is disposed in the second part of the lumen of the shaft, and the second wire is disposed in the lumen of the third coil, and
the protection tube is disposed in the lumen of the shaft so that the leaf spring, the first coil, the second coil, and the third coil are placed in the lumen of the protection tube.

15. The catheter according to claim 1, wherein the first coil is fixed to the leaf spring by welding, soldering, bonding, or crimping at the first fixed part and the second fixed part.

16. The catheter according to claim 1, wherein the intermediate unfixed portion has a length in its natural state in the longitudinal direction of 50% or longer of the total length L₁ of the first coil in its natural state.
